(19) United States
(12) Patent Application Publication
(10) Pub. No.: US 2009/0030235 A1
(43) Pub. Date: Jan. 29, 2009

(54) METHOD FOR FRACTIONATING STEREOISOMERIC COMPOUNDS

(75) Inventors: Christoph Jakel, Limburgerhof (DE); Rocco Paciello, Bad Durkheim (DE); Nawid Kashani-Shirazi, IIvesheim (DE); Gerd Kaibel, Lampertheim (DE)

Correspondence Address: OBLON, SPIVAK, MCCLELLAND MAIER & NEUSTADT, P.C., 1940 DUKE STREET, ALEXANDRIA, VA, 22314, US

(73) Assignee: BASF SE, Ludwigshafen (DE)

(21) Appl. No.: 12/067,372

(22) PCT Filed: Sep. 29, 2006
(86) PCT No.: PCT/EP2006/066910

§ 371 Date: Mar. 19, 2008

(30) Foreign Application Priority Data
Sep. 30, 2005 (DE) 10 2005 046 917.5
May 11, 2006 (EP) 06113819.4

(51) Int. Cl. C07C 29/74; C07C 209/88

(52) U.S. Cl. 564/461; 568/810; 568/868

(57) ABSTRACT

The present invention relates to a method for fractionating stereoisomeric compounds which have at least one alcohol and/or amino group.

The present invention relates to a method for fractionating compounds which have at least one alcohol and/or amino group.

Chiral alcohols and amines are starting materials which are in demand for example in fine chemistry and pharmaceutical chemistry. They are employed inter alia in tailored materials with nonlinear optical properties, as odorants and flavorings or as precursors for medicaments. There is a great interest in the most cost-effective provision of chiral alcohols that is possible. Chiral alcohols can be prepared and isolated in various ways. They can firstly be obtained directly or after appropriate chemical transformation from compounds which are naturally available, called the chiral pool. A further possibility for preparing chiral alcohols is by asymmetric synthesis, i.e. a reaction in which the resulting stereoisomeric products (enantiomers or diastereomers) result in unequal amounts. A further possibility is to obtain chiral alcohols starting from racemic alcohols by chiral discrimination in the sense of racemate resolution. Such a chiral discrimination is particularly advantageous when the corresponding racemic alcohol is readily available. Various methods for chiral discrimination of stereoisomeric alcohols are known. Thus, they can be subjected by means of enzymes, metal complexes or small organic molecules as catalysts to a kinetic racemate resolution in the sense of an enantiodifferentiation. However, the catalysts used are suitable only for a very limited number of alcohols and/or the high costs for producing the catalysts lead to the method being economically disadvantaged. It is moreover known to fractionate stereoisomeric alcohols by absorptive separation methods on optically active immobilized phases. In this connection, SMB chromatography (simulated moving bed chromatography) which enables stereoisomeric substance mixtures to be fractionated continuously into two different fractions is becoming increasingly important. One disadvantage of this technology derives from the relatively high costs of the chiral stationary phases to be used, and the need to use solvents from which the stereoisomers must be removed in a separate step. It is further known to fractionate racemic compounds with the aid of chiral discriminators (selectors) by distillation, specifically extractive rectification.

FR-A-2,176,621 describes a method for fractionating optical isomers by extractive distillation. This includes very generally the use of discriminators which are able to form complexes of differing stability with the optical antipodes in order to achieve a difference in relation to a physical property, specifically the boiling point.

EP-A-0970936 describes a method for fractionating optical isomers in which they are brought into contact in a countercurrent method with a discriminating liquid which comprises a discriminating agent, and with a diluent, and subjected to a fractionation of the optical isomers by absorptive separation, distillative separation or membrane separation. The chiral discriminators employed are saccharides and saccharide derivatives, especially cyclodextrins and cyclodextrin derivatives. A disadvantage of these discriminators is that they must be constructed in elaborate synthesis, leading to this method being economically disadvantaged.

The present invention is based on the object of providing a method which enables stereoisomeric compounds which have at least one alcohol, and/or amino group to be fractionated as economically as possible.

We have found that this object is achieved by a method for fractionating stereoisomeric compounds, specifically alcohols, by distillative separation in the presence of a chiral discriminator which is a metal complex which is derived from a compound of the general formula I

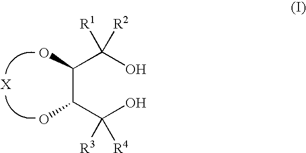

in which
R¹, R², R³ and R⁴ are independently of one another alkyl, cycloalkyl, heterocycloalkyl, aryl or hetaryl, where the alkyl radicals may have 1, 2, 3, 4 or 5 substituents selected from cycloalkyl, heterocycloalkyl, aryl, hetaryl, alkoxy, cycloalkoxy, heterocycloalkoxy, aryloxy, hetaryloxy, hydroxy, thiol, polyalkylene oxide, polyalkyleneimine, COOH, carboxylate, SO₃H, sulfonate, NE¹E², NE¹E²E³A⁻, halogen, nitro, acyl or cyano, in which E¹, E² and E³ are in each case identical or different radicals selected from hydrogen, alkyl, cycloalkyl, or aryl, and A⁻ is an anion equivalent,
and where the cycloalkyl, heterocycloalkyl, aryl and hetaryl radicals R¹, R², R³ and R⁴ may each have 1, 2, 3, 4 or 5 substituents which are selected from alkyl and the substituents mentioned above for the alkyl radicals R¹ to R⁴, or
R¹ and R² and/or R³ and R⁴ together with the carbon atom to which they are bonded are a 5- to 8-membered ring which is optionally additionally fused one, two or three times to cycloalkyl, heterocycloalkyl, aryl or hetaryl, where the ring and, if present, the fused groups may independently of one another each have one, two, three or four substituents which are selected from alkyl, cycloalkyl, heterocycloalkyl, aryl, hetaryl, hydroxy, thiol, polyalkylene oxide, polyalkyleneimine, alkoxy, halogen, COOH, carboxylate, SO₃H, sulfonate, NE⁴E⁵, NE⁴E⁵E⁶A⁻, nitro, alkoxycarbonyl, acyl or cyano, in which E⁴, E⁵ and E⁶ are in each case identical or different radicals selected from hydrogen, alkyl, cycloalkyl and aryl, and A⁻ is an anion equivalent,
X together with the oxygen atoms to which it is bonded is a 5- to 8-membered heterocycle which is optionally fused once or twice to a cycloalkyl, heterocycloalkyl, aryl and/or hetaryl, where the fused groups may independently of one another each have one, two, three or four substituents selected from alkyl, cycloalkyl, heterocycloalkyl, aryl, hetaryl, hydroxy, thiol, polyalkylene oxide, polyalkyleneimine, alkoxy, halogen, COOH, carboxylate, SO₃H, sulfonate, NE⁷E⁸, NE⁷E⁸E⁹A⁻, nitro, alkoxycarbonyl, acyl or cyano, in which E⁷, E⁸ and E⁹ are in each case identical or different radicals selected from hydrogen, alkyl, cycloalkyl and aryl, and A⁻ is an anion equivalent, and/or X may have one, two, three or four substituents which are selected from the substituents mentioned above for the fused groups, and/or X may be interrupted by 1 or 2 optionally substituted heteroatoms.

“Chiral compounds” are in the context of the present invention compounds having at least one chirality center (e.g. having at least one asymmetric atom, in particular at least one asymmetric C atom or P atom), having a chirality axis, chirality plane or screw thread. The term “chiral discriminator” comprises discriminators which have at least one chiral ligand.

“Achiral compounds” are compounds which are not chiral.

A “racemic mixture” is also called “racemate” and refers to an equimolar mixture of two chemical compounds which are related to one another as image and mirror image.

The term “racemate resolution” refers to a separation of a racemic mixture into fractions which comprise the individual compounds which are related to one another as image and mirror image in pure or at least enriched form. There is thus a racemate resolution in the sense of the invention even if non-equimolar mixtures of the components are obtained.

“Stereoisomers” are compounds of identical constitution but different atomic arrangement in three-dimensional space.

“Enantiomers” are stereoisomers which are related to one another as image to mirror image. The enantiomeric excess (ee) obtained in an asymmetric synthesis results from the following formula: ee[%]=(R−S)/(R+S)×100. R and S are the descriptors of the CIP systems for the two enantiomers and represent the absolute configuration at the asymmetric atom. The enantiopure compound (ee=100%) is also referred to as “homochiral compound”.

The method of the invention leads to products which are enriched in a particular stereoisomer. The “enantiomeric excess” (ee) achieved is usually at least 1%.

“Diastereomers” are stereoisomers which are not enantiomers of one another.

The term “alkyl” hereinafter comprises straight-chain and branched alkyl groups. Preference is given in this connection to straight-chain or branched C₁-C₂₀-alkyl, more preferably C₁-C₁₂-alkyl, particularly preferably C₁-C₈-alkyl and very particularly preferably C₁-C₆-alkyl groups. Examples of alkyl groups are in particular methyl, ethyl, propyl, isopropyl, n-butyl, 2-butyl, sec-butyl, tert-butyl, n-pentyl, 2-pentyl, 2-methylbutyl, 3-methylbutyl, 1,2-dimethylpropyl, 1,1-dimethylpropyl, 2,2-dimethylpropyl, 1-ethylpropyl, n-hexyl, 2-hexyl, 2-methylpentyl, 3-methylpentyl, 4-methylpentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl, 1-ethyl-2-methylpropyl, n-heptyl, 2-heptyl, 3-heptyl, 2-ethylpentyl, 1-propylbutyl, n-octyl, 2-ethylhexyl, 2-propylheptyl, nonyl, decyl.

The term “alkyl” also comprises substituted alkyl groups which may generally have 1, 2, 3, 4 or 5, preferably 1, 2 or 3 and particularly preferably 1 substituent selected from the groups cycloalkyl, aryl, hetaryl, halogen, NE¹E², NE¹E²E³⁺, COOH, carboxylate, —SO₃H and sulfonate.

The term “alkylene” in the sense of the present invention stands for straight-chain or branched alkanediyl groups having preferably 1 to 6, in particular 1 to 4 carbon atoms. Included therein are methylene (—CH₂—), ethylene (—CH₂—CH₂—), n-propylene (—CH₂—CH₂—CH₂—), isopropylene (—CH₂—CH(CH₃)—) etc.

The term “cycloalkyl” comprises in the sense of the present invention unsubstituted and substituted cycloalkyl groups, preferably C₃-C₈-cycloalkyl groups such as cyclopentyl, cyclohexyl or cycloheptyl, which may in the case of substitution generally have 1, 2, 3, 4 or 5, preferably 1, 2 or 3 and particularly preferably 1 substituent preferably selected from the substituents mentioned for alkyl.

The term “heterocycloalkyl” in the sense of the present invention comprises saturated, cycloaliphatic groups having in general 4 to 7, preferably 5 or 6 ring atoms, in which 1 or 2 of the ring carbon atoms are replaced by heteroatoms, preferably selected from the elements oxygen, nitrogen and sulfur, and which may optionally be substituted, where in the case of substitution these heterocycloaliphatic groups may have 1, 2 or 3, preferably 1 or 2, particularly preferably 1 substituent selected from alkyl, aryl, COOR^{f}, COO⁻M⁺ and NE¹E², preferably alkyl. Examples which may be mentioned of such heterocycloaliphatic groups are pyrrolidinyl, piperidinyl, 2,2,6,6-tetramethylpiperidinyl, imidazolidinyl, pyrazolidinyl, oxazolidinyl, morpholidinyl, thiazolidinyl, isothiazolidinyl, isoxazolidinyl, piperazinyl-, tetrahydrothiophenyl, tetrahydrofuranyl, tetrahydropyranyl, dioxanyl.

The term “aryl” comprises in the sense of the present invention unsubstituted and substituted aryl groups, and preferably stands for phenyl, tolyl, xylyl, mesityl, naphthyl, fluorenyl, anthracenyl, phenanthrenyl or naphthacenyl, 4′-biphenylyl, 3′-biphenylyl, 2′-biphenyl particularly preferably phenyl, 4′-biphenyl or naphthyl, where these aryl groups in the case of substitution may generally have 1, 2, 3, 4 or 5, preferably 1, 2 or 3 and particularly preferably 1 substituent selected from the groups alkyl, alkoxy, carboxyl, carboxylate, —SO₃H, sulfonate, NE¹E², alkylene-NE¹E², nitro, cyano or halogen.

The term “hetaryl” comprises in the sense of the present invention unsubstituted or substituted heterocycloaromatic groups, preferably the groups pyridyl, quinolinyl, acridinyl, pyridazinyl, pyrimidinyl, pyrazinyl, pyrrolyl, imidazolyl, pyrazolyl, indolyl, purinyl, indazolyl, benzotriazolyl, 1,2,3-triazolyl, 1,3,4-triazolyl and carbazolyl, where these heterocycloaromatic groups may in the case of substitution generally have 1, 2 or 3 substituents selected from the groups alkyl, alkoxy, acyl, carboxyl, carboxylate, —SO₃H, sulfonate, NE¹E², alkylene-NE¹E² or halogen. The above explanations of the terms “alkyl”, “cycloalkyl”, “aryl”, “heterocycloalkyl” and “hetaryl” apply correspondingly to the terms “alkoxy”, “cycloalkoxy”, “aryloxy”, “heterocycloalkoxy” and “hetaryloxy”.

The term “acyl” stands in the sense of the present invention for alkanoyl or aroyl groups having in general 2 to 11, preferably 2 to 8 carbon atoms, for example for the acetyl, propanoyl, butanoyl, pentanoyl, hexanoyl, heptanoyl, 2-ethylhexanoyl, 2-propylheptanoyl, benzoyl or naphthoyl group.

The radicals E¹ to E¹² are selected independently of one another from hydrogen, alkyl, cycloalkyl and aryl. The groups NE¹E², NE⁴E⁵, NE⁷E⁸ and NE¹⁰E¹¹ preferably stand for N,N-dimethylamino, N-ethyl-N-methylamino, N,N-diethylamino, N,N-dipropylamino, N,N-diisopropylamino, N,N-di-n-butylamino, N,N-di-t-butylamino, N,N-dicyclohexylamino or N,N-diphenylamino.

Halogen stands for fluorine, chlorine, bromine and iodine, preferably for fluorine, chlorine and bromine.

A cation equivalent means a singly charged cation or the fraction of a multiply charged cation which corresponds to a single positive charge. Preference is given to the use of alkali metal, especially Na⁺, K⁺, Li⁺ ions or onium ions such as ammonium, mono-, di-, tri-, tetraalkylammonium, phosphonium, tetraalkylphosphonium or tetraaryl-phosphonium ions.

In a preferred embodiment, the substituents R¹ and R² and/or R³ and R⁴ in compounds of the general formula I are groups which are not connected together. R¹, R², R³ and R⁴ are preferably selected independently of one another from groups of the formulae II.a to II.c

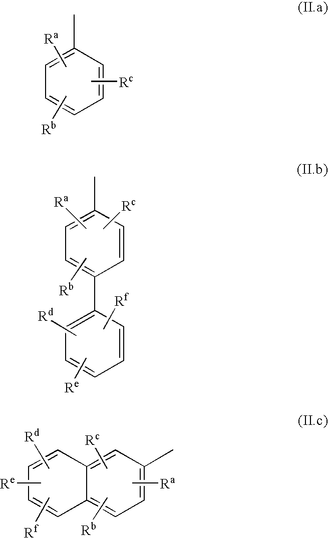

R^{a}, R^{b}, R^{c}, R^{d}, R^{e} and R^{f} are independently of one another hydrogen, C₁-C₄-alkyl, C₁-C₄-alkoxy, acyl, halogen, trifluoromethyl, C₁-C₄-alkoxycarbonyl or carboxyl.

In a preferred embodiment, the radicals R¹, R², R³ and R⁴ are selected from radicals of the formula II.a in which R^{a}, R^{b} and R^{c} are independently of one another hydrogen, C₁-C₄-alkyl, particularly preferably methyl, ethyl, n-propyl, isopropyl, n-butyl or tert-butyl, C₁-C₄-alkoxy, preferably methoxy, ethoxy, n-propoxy, isopropoxy, n-butoxy, tert-butoxy, aryl, preferably phenyl, halogen, preferably fluorine or chlorine, trifluoromethyl, methoxycarbonyl or ethoxycarbonyl. In a preferred embodiment, R^{a}, R^{b} and R^{c} are hydrogen.

In a further preferred embodiment, the radicals R¹, R², R³ and R⁴ are selected from radicals of the formula II.b in which R^{a}, R^{b}, R^{c}, R^{d}, R^{e} and R^{f} are independently of one another hydrogen, C₁-C₄-alkyl, particularly preferably methyl, ethyl, n-propyl, isopropyl, n-butyl or tert-butyl, C₁-C₄-alkoxy, preferably methoxy, ethoxy, n-propoxy, isopropoxy, n-butoxy, tert-butoxy, halogen, preferably fluorine or chlorine, trifluoromethyl, methoxycarbonyl or ethoxycarbonyl. Preferably one, two or three of the radicals R^{a} to R^{f} are selected independently of one another from C₁-C₄-alkyl, particularly preferably methyl, ethyl, n-propyl, isopropyl, n-butyl or tert-butyl, C₁-C₄-alkoxy, preferably methoxy, ethoxy, n-propoxy, isopropoxy, n-butoxy, tert-butoxy, halogen, preferably fluorine or chlorine, trifluoromethyl, methoxycarbonyl or ethoxycarbonyl, and the remaining radicals R^{a} to R^{f} are hydrogen. In a preferred embodiment, R^{a}, R^{b}, R^{c}, R^{d}, R^{e} and R^{f} are hydrogen.

In a further preferred embodiment, the radicals R¹, R², R³ and R⁴ are selected from radicals of the formula II.c, in which R^{a}, R^{b}, R^{c}, R^{d}, R^{e} and R^{f} are independently of one another hydrogen, C₁-C₄-alkyl, particularly preferably methyl, ethyl, n-propyl, isopropyl, n-butyl or tert-butyl, C₁-C₄-alkoxy, preferably methoxy, ethoxy, n-propoxy, isopropoxy, n-butoxy, tert-butoxy, halogen, preferably fluorine or chlorine, trifluoromethyl, methoxycarbonyl or ethoxycarbonyl. Preferably one, two or three of the radicals R^{a} to R^{f} are selected independently of one another from C₁-C₄-alkyl, particularly preferably methyl, ethyl, n-propyl, isopropyl, n-butyl or tert-butyl, C₁-C₄-alkoxy, preferably methoxy, ethoxy, n-propoxy, isopropoxy, n-butoxy, tert-butoxy, halogen, preferably fluorine or chlorine, trifluoromethyl, methoxycarbonyl or ethoxycarbonyl, and the remaining radicals R^{a} to R^{f} are hydrogen. In a preferred embodiment, R^{a}, R^{b}, R^{c}, R^{d}, R^{e} and R^{f} are hydrogen.

It is particularly preferred for R¹, R², R³ and R⁴ all to be phenyl, all to be 4-biphenylyl or all to be 2-naphthyl.

In a further preferred embodiment, R¹ and R² and/or R³ and R⁴ together with the carbon atom to which they are bonded are a 5- to 8-membered heterocycle which is optionally additionally fused one, two or three times to cycloalkyl, heterocycloalkyl, aryl or hetaryl, where the heterocycle and, if present, the fused groups may independently of one another each have one, two, three or four substituents which are selected from alkyl, cycloalkyl, heterocycloalkyl, aryl, hetaryl, hydroxy, thiol, polyalkylene oxide, polyalkyleneimine, alkoxy, halogen, COOH, carboxylate, SO₃H, sulfonate, NE⁴E⁵, NE⁴E⁵E⁶A⁻, nitro, alkoxycarbonyl, acyl or cyano, in which E⁴, E⁵ and E⁶ are in each case identical or different radicals selected from hydrogen, alkyl, cycloalkyl and aryl, and A⁻ is an anion equivalent.

The bridging group X is preferably selected from groups of the formulae C═O, C═S and CR⁵R⁶ in which
R⁵ and R⁶ are independently of one another alkyl, cycloalkyl, heterocycloalkyl, aryl or hetaryl, where the alkyl radicals may have 1, 2, 3, 4 or 5 substituents selected from cycloalkyl, heterocycloalkyl, aryl, hetaryl, alkoxy, cycloalkoxy, heterocycloalkoxy, aryloxy, hetaryloxy, hydroxy, thiol, polyalkyleneoxide, polyalkyleneimine, COOH, carboxylate, SO₃H, sulfonate, NE¹⁰E¹¹, NE¹⁰E¹¹E¹²A⁻, halogen, nitro, acyl or cyano, in which E¹⁰, E¹¹ and E¹² are in each case identical or different radicals selected from hydrogen, alkyl, cycloalkyl, or aryl, and A⁻ is an anion equivalent,
and where the cycloalkyl, heterocycloalkyl, aryl and hetaryl radicals R⁵ and R⁶ may each have 1, 2, 3, 4 or 5 substituents which are selected from alkyl and the substituents mentioned above for the alkyl radicals R⁵ to R⁶, or

R⁵ and R⁶ together with the carbon atom to which they are bonded are a 5- to 8-membered heterocycle which optionally is additionally fused one, two or three times to cycloalkyl, heterocycloalkyl, aryl or hetaryl, where the heterocycle and, if present, the fused groups may, independently of one another each have one, two, three or four substituents which are selected from alkyl, cycloalkyl, heterocycloalkyl, aryl, hetaryl, hydroxy, thiol, polyalkylene oxide, polyalkyleneimine, alkoxy, halogen, COOH, carboxylate, SO₃H, sulfonate, NE¹³E¹⁴, NE¹³E¹⁴E¹⁵A⁻, nitro, alkoxycarbonyl, acyl or cyano, in which E⁴, E⁵ and E⁶ are in each case identical or different radicals selected from hydrogen, alkyl, cycloalkyl and aryl, and A⁻ is an anion equivalent.

R⁵ and R⁶ are preferably both C₁-C₄-alkyl and in particular both methyl.

Preferred compounds of the formula I are those selected from compounds of the formula I.1

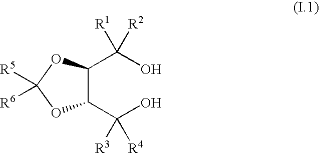

in which R¹, R², R³, R⁴, R⁵ and R⁶ have the meanings indicated previously.

In the method of the invention for fractionating stereoisomeric compounds, specifically alcohols, there is preferably use of a metal complex derived from a metal of group Ia, IIa, IIIa, IVa or Va, of group Ib to VIIIb, of the lanthanides or the actinides. The metal is preferably selected from B, Al, Ga, In, Tl, Si, Ge, Sn, Pb, As, Sb, Bi, Ti, Zr, V, Cr, Mo, W, Mn, Fe, Ru, Os, Co, Rh, Ir, Ni, Pd, Pt, Cu, Ag, Au, Zn and Cd. The metal is in particular Al, Ga, In.

If the compound to be fractionated is a stereoisomeric monoalcohol, the metal is preferably a metal which is capable of forming a stable trivalent oxidation state. These are preferably selected from Al, Ga and In. The metal is then in particular Al.

If the compound to be fractionated is a stereoisomeric compound having two functional groups which are selected from alcohol and/or amino groups, metals preferably suitable as metal are those capable of forming a stable tetravalent oxidation state. These include in particular Ti, Zr, Ge, Sn. The metal is then in particular titanium.

In this embodiment, the compounds to be fractionated are then selected in particular from diols, amino alcohols and diamines. These preferably have two hydroxyl groups or two amino groups or one hydroxyl group and one amino group in each case in the α and β position relative to one another. Suitable compounds are mentioned below.

In addition to the ligands of the formula I described above, the discriminators may also have at least one further ligand which is preferably selected from alcohols, thiols, amines, halides, alkanes, carboxylates, acetylacetonate, aryl- or alkylsulfonates, hydride, N-containing heterocycles, aromatic and heteroaromatic compounds.

The metal complex employed as chiral discriminator is preferably selected from compounds of the general formula I.a

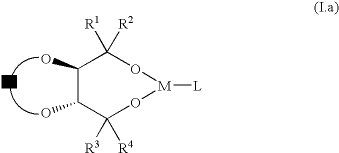

in which
R¹, R², R³, R⁴ and X have the meanings indicated previously,
M is a metal, preferably Al, and
L is selected from alkyloxy, cycloalkyloxy, aryloxy, arylalkyloxy, alkylthio, cycloalkylthio, arylthio, arylalkylthio, N,N-dialkylamino, N,N-dicycloalkylamino, N,N-diarylamino, N,N-diarylalkylamino, N-aryl-N-alkylamino, N-aryl-N-arylalkylamino, alkyl, cycloalkyl, aryl or arylalkyl.
L is preferably derived from the stereoisomeric alcohols employed for the fractionation.

In an alternative preferred embodiment, L is selected from optionally substituted phenols. Substituted phenols may preferably have 1, 2, 3 or 4 substituents which are selected from the substituents mentioned at the outset for “aryl”. In this connection, preferred phenols have at least one substituent in position 2. The substituents of the phenols are preferably selected from C₁-C₄-alkyl, particularly preferably methyl, ethyl, n-propyl, isopropyl, n-butyl or tert-butyl, C₁-C₄-alkoxy, preferably methoxy, ethoxy, n-propoxy, isopropoxy, n-butoxy, tert-butoxy, halogen, preferably fluorine or chlorine, trifluoromethyl, methoxycarbonyl or ethoxycarbonyl. Unsubstituted phenols are further preferred.

In a particularly preferred embodiment, L is selected from alcohols or phenols which have a higher boiling point than the racemate to be separated.

The metal complex is preferably prepared by employing an aluminum trialcoholate such as aluminum ethoxide (aluminum triethoxide), aluminum n-propoxide, aluminum isopropoxide, aluminum n-butoxide, aluminum sec-butoxide, aluminum tert-butoxide, aluminum phenolate, etc.

The metal complex employed as chiral discriminator is preferably selected from compounds of the general formula I.b

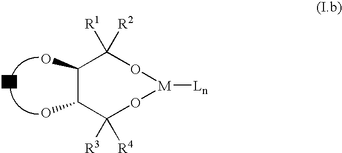

in which
n=is 1 or 2, depending on the valency of the metal M,
R¹, R², R³, R⁴ and X have the meanings indicated previously,
M is a metal, preferably Al or Ti, and
L is selected from alkyloxy, cycloalkyloxy, aryloxy, arylalkyloxy, alkylthio, cycloalkylthio, arylthio, arylalkylthio, N,N-dialkylamino, N,N-dicycloalkylamino, N,N-diarylamino, N,N-diarylalkylamino, N-aryl-N-alkylamino, N-aryl-N-arylalkylamino, alkyl, cycloalkyl, aryl or arylalkyl.
L is preferably derived from the stereoisomeric alcohols employed for the fractionation.

In an alternative preferred embodiment, L is selected from optionally substituted phenols. Substituted phenols may preferably have 1, 2, 3 or 4 substituents which are selected from the substituents mentioned at the outset for “aryl”. In this connection, preferred phenols have at least one substituent in position 2. The substituents of the phenols are preferably selected from C₁-C₄-alkyl, particularly preferably methyl, ethyl, n-propyl, isopropyl, n-butyl or tert-butyl, C₁-C₄-alkoxy, preferably methoxy, ethoxy, n-propoxy, isopropoxy, n-butoxy, tert-butoxy, halogen, preferably fluorine or chlorine, trifluoromethyl, methoxycarbonyl or ethoxycarbonyl. Unsubstituted phenols are further preferred.

In a particularly preferred embodiment, L is selected from alcohols or phenols which have a higher boiling point than the racemate to be separated.

The metal complex is preferably prepared by employing an aluminum trialcoholate such as aluminum ethoxide (aluminum triethoxide), aluminum n-propoxide, aluminum isopropoxide, aluminum n-butoxide, aluminum sec-butoxide, aluminum tert-butoxide, aluminum phenolate, etc. or a titanium tetraalcoholate such as titanium ethoxide, titanium n-propoxide, titanium isopropoxide, titanium n-butoxide, titanium sec-butoxide, titanium tert-butoxide, titanium phenolate, etc.

The metal complex employed as chiral discriminator is preferably selected from compounds of the general formula I.b1or I.b2

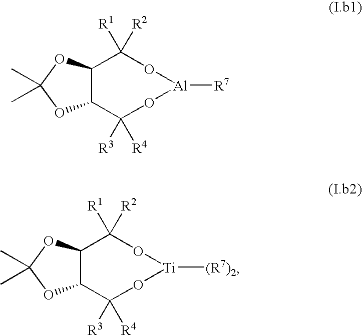

in which
R¹, R², R³ and R⁴ have the meanings indicated previously, and
R⁷ is selected from C₁-C₂₅-alkyloxy, preferably C₁-C₈-alkoxy, and phenoxy.

Some suitable discriminators are shown by way of example below

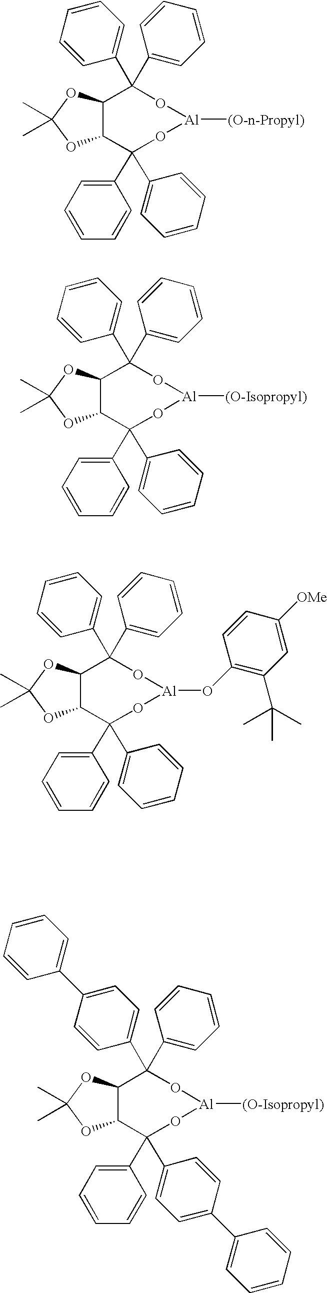

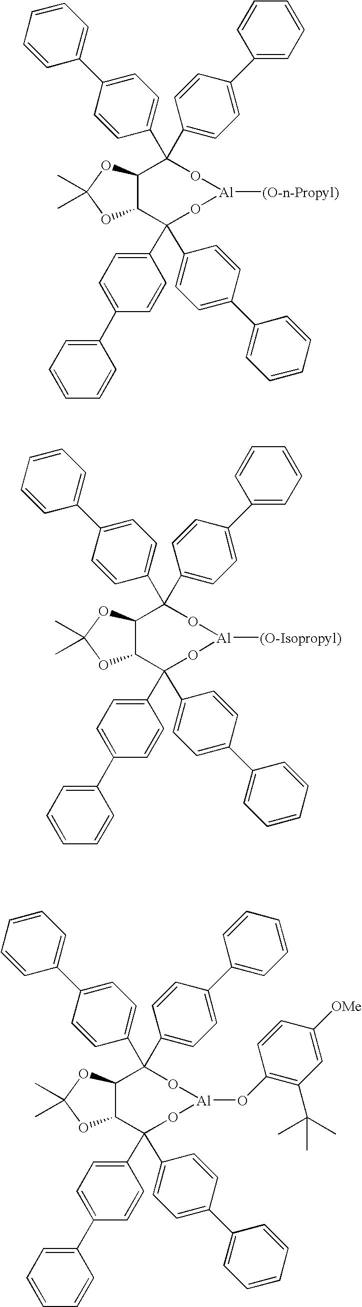

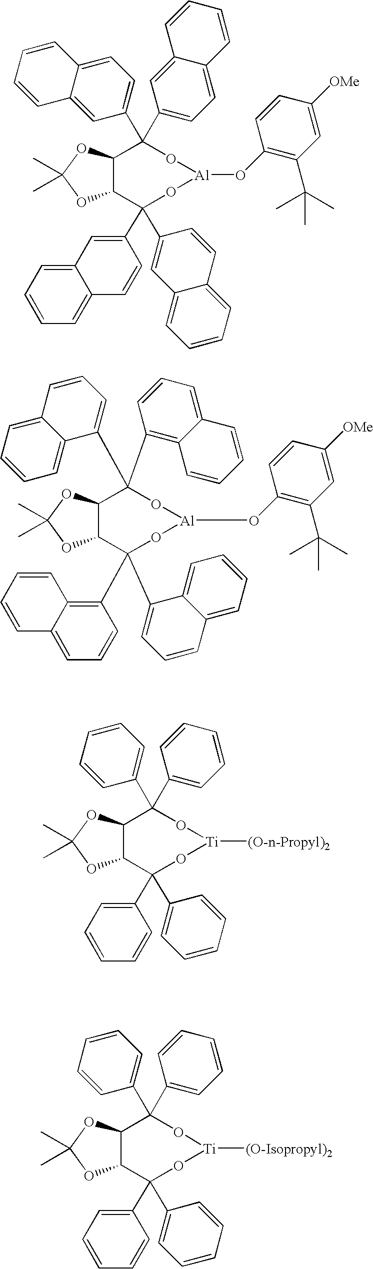

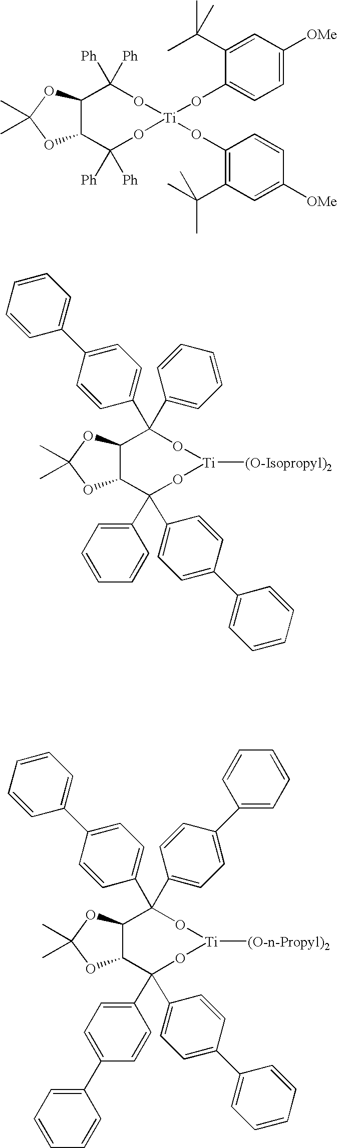

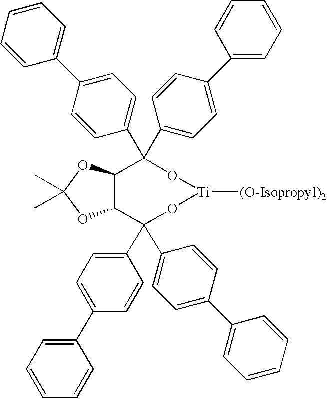

The chiral discriminators employed according to the invention lead to a boiling point difference of the stereoisomeric compounds, specifically alcohols, which have at least one alcohol and/or amino group (e.g. where enantiomeric compounds are involved) or to an increase in a preexistent boiling point difference of the stereoisomeric compounds, specifically alcohols, which have at least one alcohol and/or amino group (e.g. where diastereomeric compounds are involved).

The mixture employed for the fractionation preferably comprises two stereoisomeric compounds, specifically alcohols. The stereoisomeric compounds are preferably enantiomers of an alcohol.

The stereoisomeric compounds are particularly preferably alcohols of the general formula (III)

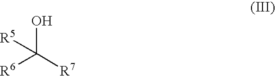

where
R⁵ and R⁶ are independently of one another selected from alkyl, cycloalkyl, heterocycloalkyl, aryl and hetaryl, each of which may optionally be substituted;
R⁷ is hydrogen or has one of the meanings given for R⁵ and R⁶, and
   where the substituents R⁵, R⁶ and R⁷ are each different from one another.

The stereoisomeric compounds of the general formula (III) are in particular the enantiomers of these compounds.

Suitable alkyl, cycloalkyl, heterocycloalkyl, aryl and hetaryl substituents are those mentioned at the outset.

Alkyl is preferably C₁-C₈-alkyl. Cycloalkyl is preferably C₃-C₈-cycloalkyl. Aryl is preferably phenyl or naphthyl, specifically phenyl.

The substituents R⁵ and R⁶ are preferably independently of one another unsubstituted or optionally halogen-substituted C₁-C₄-alkyl, specifically optionally substituted benzyl, or optionally halogen-, cyano-, nitro-, C₁-C₄-alkyl- or C₁-C₄-alkoxy-substituted aryl, in particular optionally substituted phenyl or naphthyl. R⁷ in this embodiment is preferably hydrogen.

Examples of monoalcohols which can be employed in the method of the invention and which may be mentioned are

(RS)1-phenylethanol, (RS)-2-butynol, (RS)-2-pentynol, (RS)1-phenylpropanol,
(RS)1-(4-chlorophenyl)ethanol, (RS)1-(4-chlorophenyl)propanol,
(RS)2-chloro-1-phenylethanol, (RS)3-chloro-1-phenylpropanol,
(RS)2-chloro-1-(4-chlorophenyl)ethanol, (RS)3-chloro-1-(4-chlorophenyl)propanol,
(RS)2-chloro-1-(3-chlorophenyl)ethanol, (RS)3-chloro-1-(3-chlorophenyl)propanol,
(RS)2-chloro-1-(2-chlorophenyl)ethanol, (RS)3-chloro-1-(2-chlorophenyl)propanol,
(RS)1-(4-nitrophenyl)ethanol, (RS)1-(4-nitrophenyl)propanol, (RS) 1-naphthylethanol,
(RS)1-naphthylpropanol, (RS)1-(6-methoxynaphthyl)ethanol,
(RS)1-(6-methoxynaphthyl)propanol, (RS)2-chloro-1-naphthylethanol,
(RS)3-chloro-1-naphthylpropanol, (RS)2-chloro-1-(6-methoxynaphthyl)ethanol,
(RS)3-chloro-1-(6-methoxynaphthyl)propanol, (RS )1-(4-methylpheny )ethanol,
(RS)1-(4-methylphenyl)propanol, (RS)2-chloro-1-(4-methylphenyl)ethanol,
(RS)3-chloro-1-(4-methylphenyl)propanol, (RS)1-(4-ethylphenyl)ethanol,
(RS)1-(4-ethylphenyl)propanol, (RS)2-chloro-1-(4-ethylphenyl)ethanol,
(RS)3-chloro-1-(4-ethylphenyl)propanol, (RS)1-(4-methoxyphenyl)ethanol,
(RS)1-(4-methoxyphenyl)propanol, (RS)2-chloro-1-(4-methoxyphenyl)ethanol,
(RS)3-chloro-1-(4-methoxyphenyl)propanol, (RS)1-(2-methylphenyl)ethanol,
(RS)1-(2-methylphenyl)propanol, (RS)2-chloro-1-(2-methylphenyl)ethanol,
(RS)3-chloro-1-(2-methyl phenyl)propanol, (RS)1-(2-ethylphenyl)ethanol,
(RS)1-(2-ethylphenyl )propanol, 2-chloro-1-(2-ethylphenyl)ethanol,
(RS)3-chloro-1-(2-ethylphenyl)propanol, (RS)1-(2-methoxyphenyl)ethanol,
(RS)1-(2-methoxyphenyl )propanol, 2-chloro-1-(2-methoxyphenyl)ethanol,
(RS)3-chloro-1-(2-methoxyphenyl)propanol, (RS)1-(3-methylphenyl)ethanol,
(RS)1-(3-methylphenyl)propanol, (RS)2-chloro-1-(3-methylphenyl)ethanol,
(RS)3-chloro-1-(3-methyl phenyl)propanol, (RS) 1-(3-ethylphenyl )ethanol,
(RS)1-(3-ethylphenyl)propanol, (RS)2-chloro-1-(3-ethylphenyl)ethanol,
(RS)3-chloro-1-(3-ethylphenyl)propanol, (RS )1-(3-methoxyphenyl)ethanol,
(RS)1-(3-methoxyphenyl )propanol, (RS)2-chloro-1-(3-methoxyphenyl)ethanol,
(RS)3-chloro-1-(3-methoxyphenyl)propanol and (RS)1-(1,3)-benzodioxoleethanol.

In a further preferred form, the mixture employed for the fractionation comprises stereoisomeric compounds in particular selected from diols, amino alcohols and diamines. These preferably have two hydroxyl groups or two amino groups or one hydroxyl group and one amino group in each case in α and β position relative to one another. The stereoisomeric compounds are preferably enantiomers or diastereomers.

The stereoisomeric compounds are particularly preferably compounds of the general formula (IV)

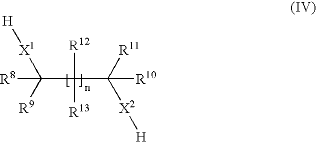

where
X¹ and X² are independently of one another O or NR¹⁴, where R¹⁴ is selected from hydrogen, alkyl, cycloalkyl and aryl, each of which may optionally be substituted;
n is 0 or 1; and
   where with the proviso that R⁸ and R⁹ are different from one another, and/or R¹⁰ and R¹¹ are different from one another, and/or R¹² and R¹³ if present are different from one another,
R⁸, R⁹, R¹⁰, R¹¹ are selected independently of one another from hydrogen, alkyl, cycloalkyl, heterocycloalkyl, aryl and hetaryl, each of which may optionally be substituted; or
   R⁸ together with R¹⁰ and the carbon atoms via which they are linked together form a 3- to 8-membered, saturated or unsaturated, nonaromatic carbocycle which is optionally substituted and may comprise further fused saturated, unsaturated or aromatic carbocycles or heterocycles; or
   if X¹ and/or X² is NR¹⁴, then R⁸ together with R¹⁴ and/or R¹⁰ together with R¹⁴ and in each case together with the atoms via which they are linked together may form an optionally substituted nitrogen-containing ring;
R¹² and R¹³ has independently of one another one of the meanings given for R⁸, R⁹, R¹⁰, R¹¹; or
   R¹² together with R⁸ and the carbon atoms via which they are linked together form a 3- to 8-membered, saturated or partly unsaturated carbocycle which is optionally substituted and may comprise further fused saturated, partly unsaturated or aromatic carbocycles or heterocycles; or
   if X¹ and/or X² is NR¹⁴, then R¹² together with R¹⁴ and/or R¹³ together with R¹⁴ and in each case together with the atoms, via which they are linked together may form an optionally substituted, nitrogen-containing ring.

Suitable alkyl, cycloalkyl, heterocycloalkyl, aryl and hetaryl substituents are those mentioned at the outset.

Alkyl is preferably C₁-C₈-alkyl. Cycloalkyl is preferably C₃-C₈-cycloalkyl. Aryl is preferably phenyl or naphthyl, specifically phenyl.

Particularly preferred compounds of the formula (IV) are those in which n is 0.

R¹² and R¹³ are further independently of one another preferably hydrogen or C₁-C₆-alkyl, where the radicals R¹² and R¹³ may be identical or different from one another. Examples which may be mentioned of stereoisomeric compounds which can be employed in the method of the invention and have two functional groups, which may be alcohol and/or amine, which are in α and β positions relative to one another are racemic mixtures of 1,2-propanediol, 3-chloro-1,2-propanediol, 3-bromo-1,2-propanediol, 1,2-butanediol, 2,3-butanediol, 1,4-dichloro-2,3-butanediol, 2-methyl-2,3-butanediol, 1,2-pentanediol, 1,2-cyclopentanediol, trans-1,2-cyclopentanediol, 1,2-hexanediol, 1,2-cyclohexanediol, trans-1,2-cyclohexanediol, 3,3-dimethyl-1,2-butanediol, 1,2-heptanediol, 2,3-heptanediol, 1,2-cycloheptanediol, trans-1,2-cycloheptanediol, 1,2-octanediol, 7-octene-1,2-diol, 1-phenyl-1,2-ethanediol, 1-phenyl-1,2-propanediol, 2-phenyl-1,2-propanediol, 1-isopropenyl-4-methyl-1,2-cyclohexanediol, 1,2,3,4-tetrahydro-2,3-naphtalenediol, 2-amino-3-hydroxybutanol (threoninol), 1-amino-2-propanol, 2-amino-1-propanol, 1-amino-2-butanol, 2-amino-1-butanol, 2-amino-2-methyl-1-propanol, 2-amino-3-methyl-1-butanol (valinol), 2-amino-1-pentanol, 2-amino-4-methylmercapto-1-butanol (methioninol), 2-amino-3-methyl-1-pentanol (isoleucinol), 2-amino-4-methyl-1-pentanol (leucinol), 2-amino-3,3-dimethyl-1-butanol (tert-leucinol), 2-amino-1-hexanol, 2-aminocyclohexanol, 2-ethylamino-1-butanol, 2-amino-1-phenylethanol, 2-amino-2-phenylethanol (α-phenylglycinol), 2-amino-3-phenyl-1-propanol (phenylalanol), 2-amino-1-phenyl-1-propanol, 2-amino-3-(p-chlorophenyl)-1-propanol, 2-benzylamino-1-propanol, 2-amino-2-methyl-3-phenyl-1-propanol, 1-amino-2-indanol, 2-amino-1-indanol, 3-amino-1,2,3,4-tetrahydro-2-naphthol, 2-amino-3-(a-imidazolyl)propanol (histidinol), 2-(hydroxymethyl)pyrrolidine (prolinol), 1,2-propylenediamine, 2-methyl-1,2-propanediamine, 1,2-cyclopentanediamine, trans-1,2-cyclopentanediamine, 1,2-cyclohexanediamine, trans-1,2-cyclohexanediamine, 1,1-di(4-anisyl)-2-iso-propylethylenediamine (daipen), 1,3-butanediol, 1,3-pentanediol, 2,2,4-trimethyl-1,3-pentanediol, 1,3-cyclohexanediol, 2-methyl-2,4-pentanediol, 2,4-pentanediol, 3-methyl-2,4-pentanediol, 1,3-cyclohexanediamine and 3-amino-2-methyl-1-phenyl-1-propanol.

The method of the invention is carried out by the stereoisomers to be fractionated being brought into contact with a chiral discriminator as described previously and subjected simultaneously and/or subsequently to a distillative separation.

The boiling point difference induced by the discriminators employed according to the invention between the stereoisomeric compounds to be fractionated makes fractionation by distillation possible. This can take place in principle by conventional distillation methods known to the skilled worker. Suitable apparatuses for distillative workup comprise distillation columns such as tray columns which may be equipped with bubble caps, perforated plates, perforated trays, valves, side offtakes, etc., evaporators, such as thin-film evaporators, Sambay evaporators etc. and combinations thereof.

It is, of course, also possible to employ an inert solvent for bringing the discriminator into contact with the stereoisomeric compounds to be fractionated and/or for the separation. The preferred solvent in this connection is one which makes it possible to carry out the distillation, as described in detail below, as extractive distillation. Examples of solvents which are ordinarily suitable are aromatic compounds such as toluene and xylenes, hydrocarbons or mixtures of hydrocarbons. Also suitable are halogenated, in particular chlorinated, hydrocarbons or aromatic compounds, such as 2-chloronaphthalene. Further suitable solvents are esters of aliphatic or aromatic carboxylic acids with alkanols, for example Texanol® (2,2,4-trimethyl-1,3-pentanediol monobutyrate) or palatinols. Suitable solvents are moreover ethers, preferably diaryl ethers, such diphenyl ether, and dialkyl ethers, such as di-n-butyl ether or polyethers, for example Lutrols® (polyethylene glycol). Suitable solvents are moreover carboxamides such as N,N-dialkylcarboxamides, e.g. dimethylformamide and dimethylacetamide, and dimethylacetanilide or carbolactams such as N-alkylcarbolactams, e.g. N-methylpyrrolidone. Also suitable are sulfur-containing solvents such as dimethyl sulfoxide or sulfolane. It is also possible to use industrial white oils, thermal oils and heat-transfer fluids such as Marlotherms®, Dowtherms®, Therminol® as solvents. It is also possible to employ ketones such as cyclohexanone etc. It is also possible to use as solvents so-called “ionic liquids”. These are liquid salts, for example N,N′-dialkylimidazolium salts such as N-butyl-N′-methylimidazolium salts, tetraalkyl-ammonium salts such as tetra-n-butylammonium salts, N-alkylpyridinium salts such as n-butylpyridinium salts, tetraalkylphosphonium salts such as trishexyl(tetradecyl)-phosphonium salts, e.g. the tetrafluoroborates, acetates, tetrachloroaluminates, hexafluorophosphates, chlorides and tosylates. The racemate to be separated may itself serve as a further suitable solvent.

In a preferred embodiment of the method of the invention, the separation takes place by extractive distillation. Extractive distillation is a method known in principle for fractionating mixtures of components which differ only slightly in their relative volatilities or which boil azeotropically. Extractive distillation is carried out by adding an additional (selective) solvent, also referred to as extractant, which preferably has a higher boiling point than the mixture to be fractionated.

The additional solvent preferably employed for the extractive distillation has a boiling point at the pressure under which the fractionation takes place which is at least 5° C., particularly preferably at least 10° C., higher than the boiling point of the highest-boiling stereoisomer.

Additional solvents suitable for the extractive distillation are those mentioned previously.

The additional solvent preferably employed is one in which the chiral discriminator employed has, under the fractionation conditions, a solubility of at least 20 g/l, in particular of at least 50 g/l.

The method can be carried out under atmospheric pressure or under reduced or elevated pressure. The method is preferably carried out under reduced pressure in such a way that the temperature at the top of the column is less than 200° C., preferably less than 150° C., particularly preferably less than 100° C.

The distillation according to the invention normally takes place under a pressure in the range from 0.01 to 900 mbar, preferably from 0.1 to 500 mbar.

The extractive distillation in the presence of the discriminator can be repeated more than once. The number of repetitions depends on the desired purity of the enantiomeric or diastereomeric products and can be determined by the skilled worker by methods known per se.

The invention is explained in more detail by means of the following, non-restrictive examples.

### EXAMPLES

### Example 1

Separation of racemic 1-phenylethanol using the chiral selector generated from (4R,5R)-(bishydroxydiphenylmethyl)-2,2-dimethyl-1,3-dioxolane and Al(O-isopropyl)₃

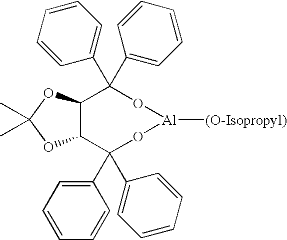

50 ml of diphenyl ether are added to 15 mmol (7.0 g) of (4R,5R)-bis(hydroxydiphenylmethyl)-2,2-dimethyl-1,3-dioxolane and 15 mmol (3.1 g) of aluminum triisopropoxide in a glove box. The resulting suspension is stirred at 90° C. under protective gas for 15 min. Subsequently, 30 mmol (3.7 g) of racemic 1-phenylethanol are added, stirring is continued for 90 min and then the resulting isopropanol is distilled off under about 20 mbar. Then, at 90° C. under 1 mbar, about 1 ml of phenylethanol is distilled off. Examination of the sample by chiral GC shows a composition of 58% (R)-1-phenylethanol and 42% (S)-1-phenylethanol. After hydrolysis of the distillation residue, the 1-phenylethanol remaining in the residue is examined by chiral GC. A composition of 46.6% (R)-phenylethanol and 53.4% (S)-phenylethanol is found.
GC: Column switching with precolumn: 25 m*0.25 mm Optioma-1 (Macherey & Nagel)
FD=0.5 μm, and chiral column: 30 m*0.25 mm BGB174S (BGB-Analytikvertrieb)
FD=0.25 μm; oven temp.: 115° C. 12′, 5°/*, 200° C.; precolumn: 1.4 bar H₂, chiral column: 1.1 bar H₂; column switching: at 1.9 min to chiral column; RT of R-1-phenylethanol=9.4 min; RT of S-1-phenylethanol=9.8 min.

### Example 2

Separation of racemic 1-phenylethanol using the chiral selector generated from (4R,5R)-bis(hydroxydi(4′-biphenylyl)methyl)-2,2-dimethyl-1,3-dioxolane and Al(O-isopropyl)₃

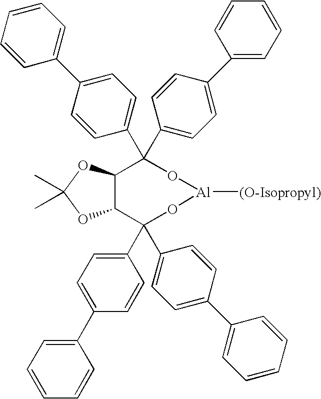

50 ml of diphenyl ether are added to 15 mmol (7.0 g) of (4R,5R)-bis(hydroxydi(4′-bi-phenylyl)methyl)-2,2-dimethyl-1,3-dioxolane and 15 mmol (3.1 g) of aluminum triisopropoxide in a glove box. The resulting suspension is stirred at 90° C. under protective gas for 15 min. Subsequently, 30 mmol (3.7 g) of racemic 1-phenylethanol are added and about 1 ml of phenylethanol is distilled off. Examination of the sample by chiral GC shows a composition of 53.4% (R)-1-phenylethanol and 46.6% (S)-1-phenylethanol.

GC: Column switching with precolumn: 25 m*0.25 mm Optioma-1 (Macherey & Nagel)
FD=0.5 μm, and chiral column: 30 m*0.25 mm BGB174S (BGB-Analytikvertrieb)
FD=0.25 μm; oven temp.: 115° C. 12′, 5°/*, 200° C.; precolumn: 1.4 bar H₂, chiral column: 1.1 bar H₂; column switching: at 1.9 min to chiral column; RT of R-1-phenylethanol=9.4 min; RT of S-1-phenylethanol=9.8 min.

### Example 3

Separation of rac.-1,2-propanediol using the chiral selector generated from (4R,5R)-bis(hydroxydiphenylmethyl)-2,2-dimethyl-1,3-dioxolane and Ti(O-isopropyl)₄

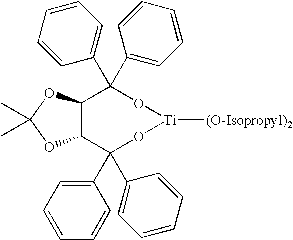

6.5 mmol (3.0 g) of (4R,5R)-bis(hydroxydiphenylmethyl)-2,2-dimethyl-1,3-dioxolane and 6.5 mmol (1.83 g) of titanium tetraisopropoxide are put into 20 ml of diphenyl ether in a glove box. The resulting yellow solution is stirred under protective gas at 90° C. for 60 min. This is followed by addition of 13 mmol (0.98 g) of rac.-1,2-propanediol and stirring for a further 10 min, and then the resulting isopropanol is distilled out under about 20 mbar. Subsequently, about 0.75 g of a mixture of diphenyl ether and 1,2-propanediol is distilled out at 1 mbar and 90° C. The enantiomeric excess determined by gas chromatography (Chirasil Dex CB, TFA derivatization) for (S)-1,2-propanediol is 16%.

### Example 4

Separation of rac.-1,2-propanediol using the chiral selector generated from (4R,5R)-bis(hydroxydiphenylmethyl)-2,2-dimethyl-1,3-dioxolane and Ti(O-isopropyl)₄

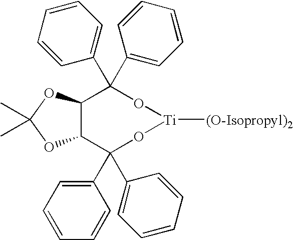

6.5 mmol (3.0 g) of (4R,5R)-bis(hydroxydiphenylmethyl)-2,2-dimethyl-1,3-dioxolane and 6.5 mmol (1.83 g) titanium tetraisopropoxide are put into 20 ml of diphenyl ether in a glove box. The resulting yellow solution is stirred under protective gas at 90° C. for 60 min. This is followed by addition of 26 mmol (1.96 g) of rac.-1,2-propanediol and stirring for a further 10 min, and then the resulting isopropanol is distilled out under about 20 mbar. Subsequently, about 0.75 g of a mixture of diphenyl ether and 1,2-propanediol is distilled out at 1 mbar and 90° C. The enantiomeric excess determined by gas chromatography (Chirasil Dex CB, TFA derivatization) for (S)-1,2-propanediol is 7.4%.

### Example 5

Separation of rac.-2-amino-1-propanol using the chiral selector generated from (4R,5R)-bis(hydroxydiphenylmethyl)-2,2-dimethyl-1,3-dioxolane and Ti(O-isopropyl)₄

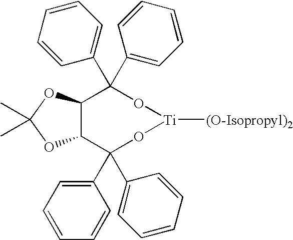

6.5 mmol (3.0 g) of (4R,5R)-bis(hydroxydiphenylmethyl)-2,2-dimethyl-1,3-dioxolane and 6.5 mmol (1.83 g) of titanium tetraisopropoxide are put into 20 ml of diphenyl ether in a glove box. The resulting yellow solution is stirred under protective gas at 90° C. for 60 min. This is followed by addition of 13 mmol (0.97 g) of rac.-2-amino-1-propanol and stirring for a further 10 min, and then the resulting isopropanol is distilled out under about 20 mbar. Subsequently, about 0.75 g of a mixture of diphenyl ether and 2-amino-1-propanol is distilled out at 1 mbar and 90° C. The enantiomeric excess determined by gas chromatography (Chirasil Dex CB, TFA derivatization) for (S)-2-amino-1-propanol is 6.3%.

### Example 6

Separation of rac.-1-phenylethanol using a chiral selector generated from (4R,5R)-bis(hydroxydiphenylmethyl)-2,2-dimethyl-1,3-dioxolane and Al(O-isopropyl)₃ (selector: alcohol ratio 0.5:1)

6.4 mmol (3.0 g) of (4R,5R)-bis(hydroxydiphenylmethyl)-2,2-dimethyl-1,3-dioxolane and 6.4 mmol (1.31 g) of aluminum triisopropoxide are put into 20 ml of diphenyl ether in a glove box. The resulting suspension is stirred under protective gas at 90° C. for 60 min. This is followed by addition of 12.8 mmol (1.56 g) of racemic 1-phenylethanol and stirring for a further 10 min. The resulting isopropanol is then distilled out under about 20 mbar. Subsequently, 1.2 g of a mixture of (R)-1-phenylethanol and (S)-1-phenylethanol are obtained as distillate at 1 mbar and 90° C. The enantiomeric excess determined by chiral gas chromatography (BGB174S, BGB-Analytikvertrieb) for (R)-1-phenylethanol was 12.0%.

### Example 7

Separation of rac.-1-phenylethanol using a chiral selector generated from (4R,5R)-bis(hydroxydiphenylmethyl)-2,2-dimethyl-1,3-dioxolane and Al(O-isopropyl)₃ (selector: alcohol ratio 2:1)

9.9 mmol (4.6 g) of (4R,5R)-bis(hydroxydiphenylmethyl)-2,2-dimethyl-1,3-dioxolane and 9.9 mmol (2.02 g) of aluminum triisopropoxide are put into 30 ml of diphenyl ether in a glove box. The resulting suspension is stirred under protective gas at 90° C. for 60 min. This is followed by addition of 4.95 mmol (0.60 g) of racemic 1-phenylethanol and stirring for a further 10 min, and then the resulting isopropanol is distilled out under about 20 mbar. Subsequently, 1.0 g of a mixture of (R)-1-phenylethanol and (S)-1-phenylethanol is obtained as distillate at 1 mbar and 90° C. The enantiomeric excess determined by gas chromatography (BGB174S, BGB-Analytikvertrieb) for (R)-1-phenylethanol was 33.8% ee.

### Example 8

Separation of rac.-1-phenylethanol using a chiral selector generated from (4R,5R)-bis(hydroxydiphenylmethyl)-2,2-dimethyl-1,3-dioxolane and Al(O-isopropyl)₃ (selector: alcohol ratio 3:1)

39.0 mmol (18.0 g) of (4R,5R)-bis(hydroxydiphenylmethyl)-2,2-dimethyl-1,3-dioxolane and 39.0 mmol (7.96 g) of aluminum triisopropoxide are put into 60 ml of diphenyl ether in a glove box. The resulting suspension is stirred under protective gas at 90° C. for 60 min. This is followed by addition of 13 mmol (1.56 g) of racemic 1-phenylethanol and stirring for a further 10 min, and then the resulting isopropanol is distilled out under about 20 mbar. Subsequently, 2.35 g of a mixture of (R)-1-phenylethanol and (S)-1-phenylethanol is obtained as distillate at 1 mbar and 90° C. The enantiomeric excess determined by gas chromatography (BGB174S, BGB-Analytikvertrieb) for (R)-1-phenylethanol was 14.8% ee.

### Example 9

Separation of rac.-1-phenylethanol using a chiral selector generated from (4R,5R)-bis(hydroxydiphenylmethyl)-2,2-dimethyl-1,3-dioxolane and Al(O-isopropyl)₃ (selector: alcohol ratio 4:1)

52.0 mmol (24.0 g) of (4R,5R)-bis(hydroxydiphenylmethyl)-2,2-dimethyl-1,3-dioxolane and 52.0 mmol (10.5 g) of aluminum triisopropoxide are put into 80 ml of diphenyl ether in a glove box. The resulting suspension is stirred under protective gas at 90° C. for 60 min. This is followed by addition of 13 mmol (1.56 g) of racemic 1-phenylethanol and stirring for a further 10 min, and then the resulting isopropanol is distilled out under about 20 mbar. Subsequently, 3.7 g of a mixture of (R)-1-phenylethanol and (S)-1-phenylethanol is obtained as distillate at 1 mbar and 90° C. The enantiomeric excess determined by gas chromatography (BGB174S, BGB-Analytikvertrieb) for (R)-1-phenylethanol was 13.4% ee.

### Example 10

Separation of rac.-4-methylphenylethanol using a chiral selector generated from (4R,5R)-bis(hydroxydiphenylmethyl)-2,2-dimethyl-1,3-dioxolane and Al(O-isopropyl)₃ (selector:alcohol ratio 1:1)

13.0 mmol (6.0 g) of (4R,5R)-bis(hydroxydiphenylmethyl)-2,2-dimethyl-1,3-dioxolane and 13.0 mmol (2.62 g) of aluminum triisopropoxide are put into 40 ml of diphenyl ether in a glove box. The resulting suspension is stirred under protective gas at 90° C. for 60 min. This is followed by addition of 13 mmol (1.75 g) of racemic 1-(4-methylphenyl)ethanol and stirring for a further 10 min. Subsequently, the resulting isopropanol is distilled out under about 20 mbar. Then, at 1 mbar and 90° C., 2.65 g of distillate are obtained. Investigation of the distillate by chiral gas chromatography (BGB-Analytikvertrieb BGB-174S) revealed an enantiomeric excess of 23.6%.

### Example 11

Separation of rac.-4-chlorophenylethanol using a chiral selector generated from (4R,5R)-bis(hydroxydiphenylmethyl)-2,2-dimethyl-1,3-dioxolane and Al(O-isopropyl)₃ (selector:alcohol ratio 1:1)

13.0 mmol (6.0 g) of (4R,5R)-bis(hydroxydiphenylmethyl)-2,2-dimethyl-1,3-dioxolane and 13.0 mmol (2.62 g) of aluminum triisopropoxide are put into 40 ml of diphenyl ether in a glove box. The resulting suspension is stirred under protective gas at 90° C. for 60 min. This is followed by addition of 13 mmol (2.02 g) of racemic 1-(4-chlorophenyl)ethanol and stirring for a further 10 min. Subsequently, the resulting isopropanol is distilled out under about 20 mbar. Then, at 1 mbar and 90° C., 1.4 g of a mixture of the enantiomers of 1-(4-chlorophenyl)ethanol as distillate are obtained. Investigation of the distillate by chiral gas chromatography (BGB-Analytikvertrieb BGB-174S) revealed an enantiomeric excess of 7.3%.

### Example 12

Separation of rac.-4-fluorophenylethanol using a chiral selector generated from (4R,5R)-bis(hydroxydiphenylmethyl)-2,2-dimethyl-1,3-dioxolane and Al(O-isopropyl)₃ (selector:alcohol ratio 1:1)

13.0 mmol (6.0 g) of (4R,5R)-bis(hydroxydiphenylmethyl)-2,2-dimethyl-1,3-dioxolane and 13.0 mmol (2.62 g) of aluminum triisopropoxide are put into 40 ml of diphenyl ether in a glove box. The resulting suspension is stirred under protective gas at 90° C. for 60 min. This is followed by addition of 13 mmol (1.80 g) of racemic 1-(4-fluorophenyl)ethanol and stirring for a further 10 min. Subsequently, the resulting isopropanol is distilled out under about 20 mbar. Then, at 1 mbar and 90° C., 1.25 g of a mixture of the enantiomers of 1-(4-fluorophenyl)ethanol as distillate are obtained. Investigation of the distillate by chiral gas chromatography (BGB-Analytikvertrieb BGB-174S) revealed an enantiomeric excess of 9.1%.

### Example 13

Separation of rac.-3-(trifluoromethyl)phenylethanol using a chiral selector generated from (4R,5R)-bis(hydroxydiphenylmethyl)-2,2-dimethyl-1,3-dioxolane and Al(O-isopropyl)₃ (selector:alcohol ratio 1:1)

13.0 mmol (6.0 g) of (4R,5R)-bis(hydroxydiphenylmethyl)-2,2-dimethyl-1,3-dioxolane and 13.0 mmol (2.62 g) of aluminum triisopropoxide are put into 40 ml of diphenyl ether in a glove box. The resulting suspension is stirred under protective gas at 90° C. for 60 min. This is followed by addition of 13 mmol (2.50 g) of racemic 1-(3-trifluoromethylphenyl)ethanol and stirring for a further 10 min. Subsequently, the resulting isopropanol is distilled out under about 20 mbar. Then, at 1 mbar and 90° C., 1.25 g of a mixture of the enantiomers of 1-(3-trifluoromethylphenyl)ethanol as distillate are obtained. Investigation of the distillate by chiral gas chromatography (BGB-Analytikvertrieb BGB-174S) revealed an enantiomeric excess of 27.8%.

### Example 14

Optical purification of 1-phenylethanol (28% ee) using a chiral selector generated from (4R,5R)-bis(hydroxydiphenylmethyl)-2,2-dimethyl-1,3-dioxolane and Al(O-isopropyl)₃ (selector:alcohol ratio 2:1)

26.0 mmol (12.0 g) of (4R,5R)-bis(hydroxydiphenylmethyl)-2,2-dimethyl-1,3-dioxolane and 26.0 mmol (5.25 g) of aluminum triisopropoxide are put into 60 ml of diphenyl ether in a glove box. The resulting suspension is stirred under protective gas at 90° C. for 60 min. This is followed by addition of 13 mmol (1.56 g) of phenylethanol with an (R)-1-phenylethanol enantiomeric excess of 28% ee, and stirring for a further 10 min. Subsequently, the resulting isopropanol is distilled out under about 20 mbar. Then 1.10 g of a mixture of (R)-1-phenylethanol and (S)-1-phenylethanol are obtained as distillate at 1 mbar and 90° C. Investigation of the distillate by chiral gas chromatography (BGB-Analytikvertrieb BGB-174S) revealed an (R)-1-phenylethanol enantiomeric excess of 49.1%.

### Example 15

Optical purification of 1-phenylethanol with 47% ee using a chiral selector generated from (4R,5R)-bis(hydroxydiphenylmethyl)-2,2-dimethyl-1,3-dioxolane and Al(O-isopropyl)₃ (selector:alcohol ratio 2:1)

26.0 mmol (12.0 g) of (4R,5R)-bis(hydroxydiphenylmethyl)-2,2-dimethyl-1,3-dioxolane and 26.0 mmol (5.25 g) of aluminum triisopropoxide are put into 60 ml of diphenyl ether in a glove box. The resulting suspension is stirred under protective gas at 90° C. for 60 min. This is followed by addition of 13 mmol (1.56 g) of phenylethanol with an (R)-1-phenylethanol enantiomeric excess of 47% ee, and stirring for a further 10 min. Subsequently, the resulting isopropanol is distilled out under about 20 mbar. Then 1.10 g of a mixture of (R)-1-phenylethanol and (S)-1-phenylethanol are obtained as distillate at 1 mbar and 90° C. Investigation of the distillate by chiral gas chromatography (BGB-Analytikvertrieb BGB-174S) afforded an (R)-1-phenylethanol enantiomeric excess of 59.0%.

### Example 16

Optical purification of 1-phenylethanol with 66% ee using a chiral selector generated from (4R,5R)-bis(hydroxydiphenylmethyl)-2,2-dimethyl-1,3-dioxolane and Al(O-isopropyl)₃ (selector:alcohol ratio 2:1)

26.0 mmol (12.0 g) of (4R,5R)-bis(hydroxydiphenylmethyl)-2,2-dimethyl-1,3-dioxolane and 26.0 mmol (5.25 g) of aluminum triisopropoxide are put into 60 ml of diphenyl ether in a glove box. The resulting suspension is stirred under protective gas at 90° C. for 60 min. This is followed by addition of 13 mmol (1.56 g) of 1-phenylethanol with an (R)-1-phenylethanol enantiomeric excess of 66% ee, and stirring for a further 10 min. Subsequently, the resulting isopropanol is distilled out under about 20 mbar. Then 1.10 g of a mixture of (R)-1-phenylethanol and (S)-1-phenylethanol are obtained as distillate at 1 mbar and 90° C. Investigation of the distillate by chiral gas chromatography (BGB-Analytikvertrieb BGB-174S) revealed an (R)-1-phenylethanol enantiomeric excess of 72.8%.

### Example 17

Optical purification of phenylethanol with 76% ee using a chiral selector generated from (4R,5R)-bis(hydroxydiphenylmethyl)-2,2-dimethyl-1,3-dioxolane and Al(O-isopropyl)₃ (selector:alcohol ratio 2:1)

26.0 mmol (12.0 g) of (4R,5R)-bis(hydroxydiphenylmethyl)-2,2-dimethyl-1,3-dioxolane and 26.0 mmol (5.25 g) of aluminum triisopropoxide are put into 60 ml of diphenyl ether in a glove box. The resulting suspension is stirred under protective gas at 90° C. for 60 min. This is followed by addition of 13 mmol (1.56 g) of 1-phenylethanol with an (R)-1-phenylethanol enantiomeric excess of 76% ee, and stirring for a further 10 min. Subsequently, the resulting isopropanol is distilled out under about 20 mbar. Then 1.10 g of a mixture of (R)-1-phenylethanol and (S)-1-phenylethanol are obtained as distillate at 1 mbar and 90° C. Investigation of the distillate by chiral gas chromatography (BGB-Analytikvertrieb BGB-174S) revealed an (R)-1-phenylethanol enantiomeric excess of 83.0%.

### Example 18

Optical purification of phenylethanol with 82% ee using a chiral selector generated from (4R,5R)-bis(hydroxydiphenylmethyl)-2,2-dimethyl-1,3-dioxolane and Al(O-isopropyl)₃ (selector:alcohol ratio 2:1)

26.0 mmol (12.0 g) of (4R,5R)-bis(hydroxydiphenylmethyl)-2,2-dimethyl-1,3-dioxolane and 26.0 mmol (5.25 g) of aluminum triisopropoxide are put into 60 ml of diphenyl ether in a glove box. The resulting suspension is stirred under protective gas at 90° C. for 60 min. This is followed by addition of 13 mmol (1.56 g) of 1-phenylethanol with an (R)-1-phenylethanol enantiomeric excess of 82% ee, and stirring for a further 10 min. Subsequently, the resulting isopropanol is distilled out under about 20 mbar. Then 1.10 g of a mixture of (R)-1-phenylethanol and (S)-1-phenylethanol are obtained as distillate at 1 mbar and 90° C. Investigation of the distillate by chiral gas chromatography (BGB-Analytikvertrieb BGB-174S) revealed an (R)-1-phenylethanol enantiomeric excess of 86.3%.

### Example 19

Optical purification of phenylethanol with 86% ee using a chiral selector generated from (4R,5R)-bis(hydroxydiphenylmethyl)-2,2-dimethyl-1,3-dioxolane and Al(O-isopropyl)₃ (selector:alcohol ratio 2:1)

26.0 mmol (12.0 g) of (4R,5R)-bis(hydroxydiphenylmethyl)-2,2-dimethyl-1,3-dioxolane and 26.0 mmol (5.25 g) of aluminum triisopropoxide are put into 60 ml of diphenyl ether in a glove box. The resulting suspension is stirred under protective gas at 90° C. for 60 min. This is followed by addition of 13 mmol (1.56 g) of 1-phenylethanol with an (R)-1-phenylethanol enantiomeric excess of 86% ee, and stirring for a further 10 min. Subsequently, the resulting isopropanol is distilled out under about 20 mbar. Then 1.10 g of a mixture of (R)-1-phenylethanol and (S)-1-phenylethanol are obtained as distillate at 1 mbar and 90° C. Investigation of the distillate by chiral gas chromatography (BGB-Analytikvertrieb BGB-174S) revealed an (R)-1-phenylethanol enantiomeric excess of 89.9%.

### Example 20

Optical purification of phenylethanol with 91% ee using a chiral selector generated from (4R,5R)-bis(hydroxydiphenylmethyl)-2,2-dimethyl-1,3-dioxolane and Al(O-isopropyl)₃ (selector:alcohol ratio 2:1)

26.0 mmol (12.0 g) of (4R,5R)-bis(hydroxydiphenylmethyl)-2,2-dimethyl-1,3-dioxolane and 26.0 mmol (5.25 g) of aluminum triisopropoxide are put into 60 ml of diphenyl ether in a glove box. The resulting suspension is stirred under protective gas at 90° C. for 60 min. This is followed by addition of 13 mmol (1.56 g) of 1-phenylethanol with an (R)-1-phenylethanol enantiomeric excess of 91% ee, and stirring for a further 10 min. Subsequently, the resulting isopropanol is distilled out under about 20 mbar. Then 1.10 g of a mixture of (R)-1-phenylethanol and (S)-1-phenylethanol are obtained as distillate at 1 mbar and 90° C. Investigation of the distillate by chiral gas chromatography (BGB-Analytikvertrieb BGB-174S) revealed an (R)-1-phenylethanol enantiomeric excess of 92.4%.

### Example 21

Optical purification of 1-phenylethanol with 92.5% ee using a chiral selector generated from (4R,5R)-bis(hydroxydiphenylmethyl)-2,2-dimethyl-1,3-dioxolane and Al(O-isopropyl)₃ (selector:alcohol ratio 2:1)

26.0 mmol (12.0 g) of (4R,5R)-bis(hydroxydiphenylmethyl)-2,2-dimethyl-1,3-dioxolane and 26.0 mmol (5.25 g) of aluminum triisopropoxide are put into 60 ml of diphenyl ether in a glove box. The resulting suspension is stirred under protective gas at 90° C. for 60 min. This is followed by addition of 13 mmol (1.56 g) of 1-phenylethanol with an (R)-1-phenylethanol enantiomeric excess of 92.5% ee, and stirring for a further 10 min. Subsequently, the resulting isopropanol is distilled out under about 20 mbar. Then 1.10 g of a mixture of (R)-1-phenylethanol and (S)-1-phenylethanol are obtained as distillate at 1 mbar and 90° C. Investigation of the distillate by chiral gas chromatography (BGB-Analytikvertrieb BGB-174S) revealed an (R)-1-phenylethanol enantiomeric excess of 94.7%.

### Example 22

Optical purification of phenylethanol with 94.2% ee using a chiral selector generated from (4R,5R)-bis(hydroxydiphenylmethyl)-2,2-dimethyl-1,3-dioxolane and Al(O-isopropyl)₃ (selector:alcohol ratio 2:1)

26.0 mmol (12.0 g) of (4R,5R)-bis(hydroxydiphenylmethyl)-2,2-dimethyl-1,3-dioxolane and 26.0 mmol (5.25 g) of aluminum triisopropoxide are put into 60 ml of diphenyl ether in a glove box. The resulting suspension is stirred under protective gas at 90° C. for 60 min. This is followed by addition of 13 mmol (1.56 g) of 1-phenylethanol with an (R)-1-phenylethanol enantiomeric excess of 94.2% ee, and stirring for a further 10 min. Subsequently, the resulting isopropanol is distilled out under about 20 mbar. Then 1.10 g of a mixture of (R)-1-phenylethanol and (S)-1-phenylethanol are obtained as distillate at 1 mbar and 90° C. Investigation of the distillate by chiral gas chromatography (BGB-Analytikvertrieb BGB-174S) revealed an (R)-1-phenylethanol enantiomeric excess of 95.4%.

### Example 23

Separation of rac.-trans-1,2-diaminocyclohexane using a chiral selector generated from (4R,5R)-bis(hydroxydiphenylmethyl)-2,2-dimethyl-1,3-dioxolane and Ti(O-isopropyl)₄

6.5 mmol (3.0 g) of (4R,5R)-bis(hydroxydiphenylmethyl)-2,2-dimethyl-1,3-dioxolane and 6.5 mmol (1.83 g) of titanium tetraisopropoxide are put into 20 ml of diphenyl ether in a glove box. The resulting yellow solution is stirred under protective gas at 90° C. for 60 min. This is followed by addition of 26 mmol (2.94 g) of racemic trans-1,2-diaminocyclohexane and stirring for a further 10 min, and then the resulting isopropanol is distilled out under about 20 mbar. Subsequently, about 0.8 g of a mixture of diphenyl ether and 1,2-diaminocyclohexane is distilled out at 1 mbar and 90° C. The enantiomeric excess determined by gas chromatography (Chirasil Dex CB, TFA derivatization) for (S,S)-1,2-trans-diaminocyclohexane was 12.9%.

### Example 24

Separation of rac.-1,2-propanediol using a chiral selector generated from (4R,5R)-bis(hydroxydiphenylmethyl)-2,2-dimethyl-1,3-dioxolane and Ti(O-isopropyl)₄ in the presence of 2-tert-butyl-4-methoxyphenol and 3-tert-butyl-4-methoxyphenol

6.5 mmol (3.0 g) of (4R,5R)-bis(hydroxydiphenylmethyl)-2,2-dimethyl-1,3-dioxolane, 6.5 mmol (1.83 g) of titanium tetraisopropoxide and 13 mmol (2.32 g) of a mixture of 2-tert-butyl-4-methoxyphenol and 3-tert-butyl-4-methoxyphenol are put into 20 ml of diphenyl ether in a glove box. The resulting orange solution is stirred under protective gas at 90° C. for 60 min. This is followed by addition of 13 mmol (0.99 g) of rac.-1,2-propanediol and stirring for a further 10 min. The resulting isopropanol is then distilled out under about 20 mbar. Subsequently, about 1.0 g of a mixture of diphenyl ether and 1,2-propanediol is distilled out at 1 mbar and 90° C. The enantiomeric excess determined by gas chromatography (BGB-174S, TFA derivatization) for (S)-1,2-propanediol was 9.2%.

### Example 25

Separation of rac.-1,2-propanediol using a chiral selector generated from (4R,5R)-bis(hydroxydiphenylmethyl)-2,2-dimethyl-1,3-dioxolane and Ti(O-isopropyl)₄ in the presence of didodecylamine

6.5 mmol (3.0 g) of (4R,5R)-bis(hydroxydiphenylmethyl)-2,2-dimethyl-1,3-dioxolane, 6.5 mmol (1.83 g) of titanium tetraisopropoxide and 13 mmol (4.6 g) of didodecylamine are put into 20 ml of diphenyl ether in a glove box. The resulting clear solution is stirred under protective gas at 90° C. for 60 min. This is followed by addition of 26 mmol (1.98 g) of rac.-1,2-propanediol and stirring for a further 10 min. The resulting isopropanol is then distilled out under about 20 mbar. Subsequently, about 3.15 g of a mixture of diphenyl ether and 1,2-propanediol is distilled out at 1 mbar and 90° C. The (S)-1,2-propanediol enantiomeric excess determined by gas chromatography (BGB-174S, TFA derivatization) was 17.6%.

### Example 26

Separation of rac.-1,2-propanediol using a chiral selector generated from (4R,5R)-bis(hydroxydiphenylmethyl)-2,2-dimethyl-1,3-dioxolane and Ti(O-isopropyl)₄ in the presence of dodecylamine

6.5 mmol (3.0 g) of (4R,5R)-bis(hydroxydiphenylmethyl)-2,2-dimethyl-1,3-dioxolane, 6.5 mmol (1.83 g) of titanium tetraisopropoxide and 13 mmol (2.41 g) of dodecylamine are put into 20 ml of diphenyl ether in a glove box. The resulting clear solution is stirred under protective gas at 90° C. for 60 min. This is followed by addition of 26 mmol (1.98 g) of rac.-1,2-propanediol and stirring for a further 10 min. The resulting isopropanol is then distilled out under about 20 mbar. Subsequently, 2.3 g of a mixture of diphenyl ether and 1,2-propanediol is distilled out at 1 mbar and 90° C. The (S)-1,2-propanediol enantiomeric excess determined by gas chromatography (BGB-174S, TFA derivatization) was 14.8%.

1. A method for fractionating stereoisomeric compounds by a distillative separation in the presence of a chiral discriminator which is a metal complex having at least one ligand which is derived from a compound of formula I 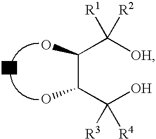
wherein
R¹, R², R³ and R⁴ are independently of one another alkyl, cycloalkyl, heterocycloalkyl, aryl or hetaryl, where the alkyl radicals may have 1, 2, 3, 4 or 5 substituents selected from the group consisting of cycloalkyl, heterocycloalkyl, aryl, hetaryl, alkoxy, cycloalkoxy, heterocycloalkoxy, aryloxy, hetaryloxy, hydroxy, thiol, polyalkylene oxide, polyalkyleneimine, COOH, carboxylate, SO₃H, sulfonate, NE¹E², NE¹E²E³A⁻, halogen, nitro, acyl and cyano, in which E¹, E² and E³ are in each case identical or different radicals selected from the group consisting of hydrogen, alkyl, cycloalkyl, and aryl, and A⁻ is an anion equivalent,
and the cycloalkyl, heterocycloalkyl, aryl and hetaryl radicals R¹, R², R³ and R⁴ may each have 1, 2, 3, 4 or 5 substituents which are selected from alkyl and the substituents for the alkyl radicals R1 to R4, or
R¹ and R² and/or R³ and R⁴ together with the carbon atom to which they are bonded are a 5- to 8-membered ring which is optionally additionally fused one, two or three times to cycloalkyl, heterocycloalkyl, aryl or hetaryl, where the ring and, if present, the fused groups may independently of one another each have one, two, three or four substituents which are selected from the group consisting of alkyl, cycloalkyl, heterocycloalkyl, aryl, hetaryl, hydroxy, thiol, polyalkylene oxide, polyalkyleneimine, alkoxy, halogen, COOH, carboxylate, SO₃H, sulfonate, NE⁴E⁵, NE⁴E⁵E⁶A⁻, nitro, alkoxycarbonyl, acyl and cyano, in which E⁴, E⁵ and E⁶ are in each case identical or different radicals selected from the group consisting of hydrogen, alkyl, cycloalkyl aryl, and A⁻ is an anion equivalent,
X together with the oxygen atoms to which it bonded is a 5- to 8-membered heterocycle which is optionally fused once or twice to a cycloalkyl, hetero-cycloalkyl, aryl and/or hetaryl, wherein the fused groups may independently of one another each have one, two, three or four substituents selected from the group consisting of alkyl, cycloalkyl, heterocycloalkyl, aryl, hetaryl, hydroxy, thiol, polyalkylene oxide, polyalkyleneimine, alkoxy, halogen, COOH, carboxylate, SO₃H, sulfonate, NE⁷E⁸, NE⁷E⁸E⁹A⁻, nitro, alkoxycarbonyl, acyl and cyano, in which E⁷, E⁸ and E⁹ are in each case identical or different radicals selected from the group consisting of hydrogen, alkyl, cycloalkyl and aryl, and A⁻ is an anion equivalent, and/or X may have one, two, three or four substituents which are selected from the substituents for the fused groups, and/or X may be interrupted by 1 or 2 optionally substituted heteroatoms.

2. The method according to claim 1, wherein the stereoisomeric compound to be fractionated is an alcohol.

3. The method according to claim 2, wherein the stereoisomeric compound to be fractionated is an alcohol of formula (III) 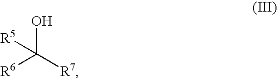
wherein
R⁵ and R⁶ are independently of one another selected from the group consisting of alkyl, cycloalkyl, heterocycloalkyl, aryl and hetaryl, each of which may optionally be substituted;
R⁷ is hydrogen or has one of the meanings given for R⁵ and R⁶, and wherein the substituents R⁵, R⁶ and R⁷ are each different from one another.

4. The method according to claim 1, where the stereoisomeric compound to be fractionated is a stereoisomeric compound having two functional groups which are selected from the group consisting of alcohol and amino groups.

5. The method according to claim 4, where the stereoisomeric compound to be fractionated is a stereoisomeric compound of formula (IV) 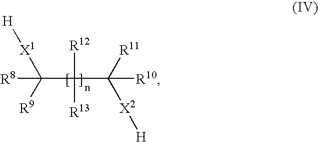
wherein
X¹ and X² are independently of one another O or NR¹⁴, where R¹⁴ is selected from the group consisting of hydrogen, alkyl, cycloalkyl and aryl, each of which may optionally be substituted;
n is 0 or 1;
with the proviso that R⁸ and R⁹ are different from one another, and/or R¹⁰ and R¹¹ are different from one another, and/or R¹² and R¹³, if present, are different from one another,
R⁸, R⁹, R¹⁰, R¹¹ are selected independently of one another from the group consisting of hydrogen, alkyl, cycloalkyl, heterocycloalkyl, aryl and hetaryl, where each of heterocycloalkyl, aryl and hetaryl radicals may optionally be substituted; or
R⁸ together with R¹⁰ and the carbon atoms via which they are linked together form a 3- to 8-membered, saturated or partly unsaturated carbocycle which is optionally substituted and may comprise further fused saturated, partly unsaturated or aromatic carbocycles or heterocycles; or
if X¹ and/or X² is NR¹⁴, then R⁸ together with R¹⁴ and/or R¹⁰ together with R¹⁴ and in each case together with the atoms via which they are linked together may form an optionally substituted nitrogen-containing ring;
R¹² and R¹³ has independently of one another one of the meanings given for R⁸, R⁹, R¹⁰, R¹¹; or
R12 together with R⁸ and the carbon atoms via which they are linked together form a 3- to 8-membered, saturated or partly unsaturated carbocycle which is optionally substituted and may comprise further fused saturated, partly unsaturated or aromatic carbocycles or heterocycles; or
if X¹ and/or X² is NR¹⁴, then R¹² together with R¹⁴ and/or R¹³ together with R¹⁴ and in each case together with the atoms, via which they are linked together may form an optionally substituted, nitrogen-containing ring.

6. The method according to claim 2, wherein the metal complex employed as chiral discriminator is selected from compounds of formula I.b 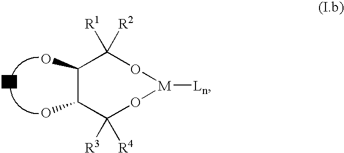
wherein
R¹, R², R³, R⁴ and X are as in claim 2,
M is a metal,
n is 1 or 2, and
L is selected independently of one another from the group consisting of alkyloxy, cycloalkyloxy, aryloxy, arylalkyloxy, alkylthio, cycloalkylthio, arylthio, arylalkylthio, N,N-dialkylamino, N,N-dicycloalkylamino, N,N-diarylamino, N,N-diarylalkylamino, N-aryl-N-alkylamino, N-aryl-N-arylalkylamino, alkyl, cycloalkyl, aryl and arylalkyl.

7. The method according to claim 6, wherein L is derived from the stereoisomeric alcohols employed for the fractionation.

8. The method according to claim 6, wherein M is a trivalent metal, and the stereoisomeric compond to be fractionated is an alcohol.

9. The method according to claim 6, wherein M is a tetravalent metal, and the stereoisomeric compound to be fractionated is a stereoisomeric compound having two functional groups which are selected from alcohol and/or amino groups.

10. The method according to claim 2, wherein the stereoisomeric compounds, are enantiomers.

11. The method according to claim 3, wherein the stereoisomeric compound having two functional groups which are selected from the group consisting of alcohol and amino groups comprises enantiomers or diastereomers.

12. The method according to claim 1, wherein the distillative separation comprises an extractive distillation.

13. The method according to claim 12, wherein an additional solvent which has a higher boiling point than the stereoisomeric compounds to be fractionated is employed for the fractionation.

14. The method according to claim 13, wherein the additional solvent employed for the fractionation has a boiling point at the pressure under which the fractionation takes place which is at least 5° C. higher than the boiling point of the highest-boiling stereoisomer.
